(19) United States
(12) Patent Application Publication
(10) Pub. No.: US 2022/0378416 A1
(43) Pub. Date: Dec. 1, 2022

(54) CURVED SUTURE NEEDLE EXTENSION

(71) Applicant: Ergosurgical Group Corp., Santa Maria, PR (US)

(72) Inventors: Luis Jose Almodovar, Santa Maria, SanJuan, PR (US); Mark A. Follman, Glen Rock, NJ (US)

(21) Appl. No.: 17/826,454

(22) Filed: May 27, 2022

Related U.S. Application Data
(60) Provisional application No. 63/194,593, filed on May 28, 2021.

(51) Int. Cl. A61B 17/06; A61B 17/04

(52) U.S. Cl. CPC A61B 17/06066; A61B 17/0491; A61B 17/06004; A61B 17/06166; A61B 2017/06019; A61B 17/00

(57) ABSTRACT

Disclosed is a suturing needle having a thread attachment portion and a ramp portion comprising a tapered portion extending from a proximal end of the needle to cover at least a portion of a suture thread swaged in the thread attachment portion. The tapered portion of the ramp portion provides a smooth transition from a diameter of the suture thread to a diameter of the needle body. The tapered transition portion allows for engaging the suture needle on rollers of a rotational needle driver in one smooth motion.

### CROSS REFERENCE TO RELATED APPLICATIONS

The present application claims the benefit of U.S. Provisional Application 63/194,593 filed 28 May 2021; which is incorporated herein by reference for all purposes.

### TECHNICAL FIELD

The present disclosure generally relates to curved suture needles.

### BACKGROUND

Suturing procedures used to close wounds or incisions consume a considerable amount of time during a surgical procedure. Generally, the instruments used in suturing procedures are the suturing material, the suturing needle, and the suturing driver. Efforts made to reduce the time and enhance the safety of the procedure have mainly focused on modifications to the needle driver.

The traditional way of driving a needle has been to clamp it down between two flat tips of a needle driver. This forms a static relationship between the needle and needle driver because the needle, once clamped down between the flat tips, cannot move relative to the driver. Moreover, needle drivers are generally designed to fit right-handed users. Thus, left-handed users have difficulties performing the suturing procedure. The right handedness of those devices further affects the capacity of left-handed surgeons to lock and unlock the drivers' locking mechanism. This increases the risks of negative outcomes for patients from sub optimal or incorrect needle driver maneuverings.

Recent advances in needle drivers address these deficiencies, including at least this static relationship of the needle and the handedness of the prior art drivers.

Current suture needles have not been designed for, and therefore cannot optimally perform, this task because they were not designed to work with new roller needle drivers. When trying to drive standard swaged needles with these new ergonomic rotational action needle drivers, the rollers generally need to be opened to move over the swage end of the needle. That is, the swage represents a step or lip on the needle that may be damaged by the rollers, or conversely, that may damage the rollers.

Accordingly, new needle designs that may enable smooth loading of a swaged needle by these new needle drivers are desirable and an object of the present disclosure.

### SUMMARY

All references, including any patents or patent applications cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents form part of the common general knowledge in the art.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e., that it will be taken to mean an inclusion of not only the listed components it directly references, but also other non-specified components or elements. This rationale will also be used when the term ‘comprised’ or ‘comprising’ is used in relation to one or more steps in a method or process.

The present disclosure provides a suturing needle comprising: a needle tip portion having a needle tip at a distal end for piercing tissue; a needle body having a distal end connected to a proximal end of the needle tip portion; a hollow thread attachment component having a distal end attached to a proximal end of the needle body, the thread attachment component comprising a viewing port, a hollow extension ramp portion having a distal end and a tapered portion at proximal end, the distal end attached to a proximal end of the thread attachment component; and a suture thread attached to the thread attachment component. In certain embodiments the distal end of the ramp has a diameter equal to the needle body diameter, and the proximal end of the ramp has a diameter equal to a suture thread diameter, and wherein the tapered portion provides a smooth transition from the proximal end of the ramp portion to the distal end thereof. In some embodiments the suture thread is attached to the thread attachment component by swaging when the suture thread is visible through the viewing port. In particular embodiments the suture thread is mono-filamentary or multi-filamentary. In other embodiments the suture thread is bioabsorbable. In further embodiments the suture thread comprises at least one region of tissue retaining structures. In yet further embodiments the tissue retaining structures are barbs. In certain embodiments the at least one region of tissue retaining structures are distal from the tapered portion of the ramp portion. In other embodiments the needle tip portion is formed to become gradually thicker from the needle tip to the proximal end thereof.

In certain embodiments the needle body is straight. In some embodiments the needle body curved. In other embodiments the needle body is formed to have a substantially circular cross-section. In further embodiments the needle body is formed to have a substantially circular cross-section having a smooth profile. In particular embodiments the needle body is formed to have a substantially circular cross-section having a non-smooth profile. In yet other embodiments the non-smooth profile comprises a plurality of equally spaced circumferential indentations extending longitudinally on the curved needle body. In certain embodiments the non-smooth profile comprises from 4 to 16 equally spaced circumferential indentations forming ridges having a convex shape. In some embodiments the circumferential indentations extend along a full length of the curved needle body. In other embodiments the circumferential indentations are angled on the curved needle body to form a spiral thereon. In yet other embodiments the spiral is right-handed or left-handed.

In further embodiments the suturing needle comprises at least two regions of circumferential indentations having different angles on the curved needle body. In additional embodiments the needle body is formed to have a substantially hexagonal or otherwise non-circular polygonal cross-section. In particular embodiments the suturing needle further comprises a hollow flexible extension component having a distal end and a proximal end, the distal end connected to the proximal end of the thread attachment component, and the proximal end attached to the distal end of the tapered extension ramp portion. In some embodiments the hollow flexible extension component has a diameter equal to the needle body diameter. In other embodiments the hollow flexible extension component comprises laser cuts. In yet other embodiments the laser cuts form a spiral. In certain embodiments the hollow flexible extension component comprises a cable. In some embodiments the cable forms a spiral. In additional embodiments the hollow flexible extension component comprises a wire. In other embodiments the wire forms a spiral.

The present disclosure also provides a method of suturing tissues using a roller needle driver, the method comprising the steps of: (a) providing a suture needle comprising: a needle tip portion having a needle tip at a distal end for piercing tissue; a needle body having a distal end connected to a proximal end of the needle tip portion; a hollow thread attachment component having a distal end attached to a proximal end of the needle body, the thread attachment component comprising a viewing port, a ramp portion having a distal end and a tapered portion at proximal end, the distal end attached to a proximal end of the thread attachment component; and a suture thread attached to the thread attachment component; (b) grasping, between rollers of the roller needle driver, the suture thread or the tapered portion of the ramp portion distal from the needle body; (c) loading the needle so that the rollers are positioned on the needle body without opening the rollers; (d) driving the needle through a tissue being subjected to suture; (e) moving the needle through the tissue without releasing the needle from the tissue; (f) grasping the suture needle at an end exiting from the tissue through which the needle was moved; and (g) repeating steps (d)-(f) until completion of the suturing process.

The system as described herein, both as to its configuration and its mode of operation will be best understood, and additional objects and advantages thereof will become apparent, by the following detailed description of a preferred embodiment taken in conjunction with the accompanying drawing.

When the word “invention” is used in this specification, the word “invention” includes “inventions”, that is, the plural of “invention”. By stating “invention”, the Applicant does not in any way admit that the present application does not include more than one patentable and non-obviously distinct invention and Applicant maintains that the present application may include more than one patentable and non-obviously distinct invention. The Applicant hereby asserts, that the disclosure of the present application may include more than one invention, and, in the event that there is more than one invention, that these inventions may be patentable and non-obvious one with respect to the other.

Further, the purpose of the accompanying abstract is to enable the U.S. Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the full scope of the application, which is measured by the claims, nor is it intended to be limiting as to the scope of the disclosure in any way.

### BRIEF DESCRIPTION OF THE DRAWINGS

All of the figures depict preferred embodiments although other embodiments are contemplated, and the present disclosure is not limited to the embodiments shown.

FIG. 1 is an illustration of a curved suture needle according to one embodiment of the present disclosure.

FIG. 2 is an illustration of a curved suture needle having a laser cut hollow flexible extension portion according to one embodiment of the present disclosure.

FIG. 3 is an illustration of a curved suture needle having a spiral wire hollow flexible extension portion according to one embodiment of the present disclosure.

FIG. 4A is an illustration of a curved suture needle having a spiral cable hollow flexible extension portion according to one embodiment of the present disclosure.

FIG. 4B is an illustration of the spiral cable hollow flexible extension portion as shown in FIG. 4A.

FIG. 4C is an illustration of a cross-section of the spiral cable hollow flexible extension portion along line A-A as shown in FIG. 4B.

FIG. 5A is an illustration of a hexagonal curved suture needle having a spiral cable hollow flexible extension portion according to one embodiment of the present disclosure.

FIG. 5B is an illustration of a cross-section of the hexagonal curved suture needle along line A-A as shown in FIG. 5A.

FIG. 6A is an illustration of a curved suture needle being fed into the rollers of a rotational driver engaging at the transition between the suture thread and the tapered ramp section of the extension according to one embodiment of the present disclosure.

FIG. 6B is an illustration of a curved suture needle being fed into the rollers of a rotational driver engaging at the extension portion according to one embodiment of the present disclosure.

FIG. 6C is an illustration of a curved suture needle being fed into the rollers of a rotational driver engaging at the needle body according to one embodiment of the present disclosure.

FIG. 7A is a curved needle having a tapered transition portion and a non-smooth profile according to one embodiment of the present disclosure.

FIG. 7B is a cross-sectional view taken along line 1-1 of the curved suture needle shown in FIG. 7A according to one embodiment of the present disclosure.

FIG. 8A, FIG. 8B and FIG. 8C are illustrations of alternative cross-sections of the suture needle having a non-smooth profile according to certain embodiments of the present disclosure.

FIG. 9 illustrates an alternative arrangement of the non-smooth surface according to certain embodiments of the present disclosure.

FIG. 10A and FIG. 10B are illustrations of a curved suture needle, shown in partial cross-section, according to certain embodiments of the present disclosure held within a rotational needle driver, wherein FIG. 10A shows the needle in the usual perpendicular orientation passing behind the rollers of the driver, and FIG. 10B shows the needle in the usual perpendicular orientation passing in front of the rollers of the driver.

FIG. 11A and FIG. 11B are illustrations of a curved suture needle according to certain embodiments of the present disclosure held within a rotational needle driver, wherein FIG. 11A shows the needle in a parallel orientation passing in front of the rollers of the driver, and FIG. 11B shows the needle in an angled orientation relative to the rollers and passing in front of the rollers of the driver.

FIG. 12 is an illustration of a curved suture needle according to certain embodiments of the present disclosure.

FIG. 13A, FIG. 13B, FIG. 13C, FIG. 13D, FIG. 13E and FIG. 13F are illustrations of a curved suture needle according to certain embodiments of the present disclosure.

FIG. 14 is an illustration of a curved suture needle according to certain embodiments of the present disclosure.

FIG. 15 is an illustration of a curved suture needle according to certain embodiments of the present disclosure.

FIG. 16 is an illustration of a curved suture needle according to certain embodiments of the present disclosure.

FIG. 17A is an illustration of a curved suture needle according to certain embodiments of the present disclosure. FIG. 17B shows an exemplary embodiment of the sleeve showing a laser-cut “dog bone” pattern.

FIG. 18A is an illustration of a curved suture needle according to certain embodiments of the present disclosure. FIG. 18B shows an exemplary embodiment of the sleeve showing a laser-cut “dog bone” pattern.

FIG. 19 is an illustration of a curved suture needle according to certain embodiments of the present disclosure.

FIG. 20 is an illustration of a curved suture needle according to certain embodiments of the present disclosure.

### DETAILED DESCRIPTION

To provide an overall understanding of the disclosure, certain illustrative embodiments and examples will now be described. However, it will be understood by one of ordinary skill in the art that the same or equivalent functions and sequences may be accomplished by different embodiments that are also intended to be encompassed within the spirit and scope of the disclosure. The compositions, apparatuses, systems and/or methods described herein may be adapted and modified as is appropriate for the application being addressed and that those described herein may be employed in other suitable applications, and that such other additions and modifications will not depart from the scope hereof.

As used in the specification and claims, the singular forms “a”, “an” and “the” include plural references unless the context clearly dictates otherwise. For example, the term “material” may include a plurality of materials unless the context clearly dictates otherwise. As used in the specification and claims, singular names or types referenced include variations within the family of said name unless the context clearly dictates otherwise.

Certain terminology is used in the following description for convenience only and is not limiting. The words “lower,” “upper,” “bottom,” “top,” “front,” “back,” “left,” “right” and “sides” designate directions in the drawings to which reference is made, but are not limiting with respect to the orientation in which the various parts of the needle or any assembly of them may be used.

Swaged suturing needles typically comprise attachment of a suture thread to an end of a needle body. These needles have been designed for use with standard needle drivers, which clamp the needle between two flat tips and hold the needle in a static position.

A new generation of devices that move the needle through the tissues by the coordinated rotation of two rollers provide a dynamic relationship between the needle and the needle driver, even when the needle is clamped between the rollers. One of the great advantages of this dynamic relationship is that the needle can be driven in multiple planes of rotation, such as perpendicular to the long axis of the driver with the tip pointing up or down (standard), parallel with the long axis of the driver with the tip pointing straight back towards the back of the device or straight forward away from the device (non-standard), and any angle in between. This dynamic relationship is desirable for the surgeon as it enables placement of the needle at non-traditional angles, permitting suturing of difficult anatomical regions or situations, such as restricted, deep and less accessible locations.

According to certain embodiments, a needle comprising a needle tip portion having a needle tip at a distal end for piercing tissue, a straight or curved trunk portion having a distal end connected to a proximal end of the needle tip portion; and a thread attachment portion connected to a proximal end of the trunk portion for connecting a suturing thread, wherein the needle tip portion is formed to become gradually thicker from the needle tip to the proximal end thereof, and wherein the trunk portion is formed to have a substantially circular cross-section having a non-smooth profile. In certain embodiments, the needle may have a smooth profile. In one or more embodiment(s), the needle comprises at least one of a circular, an oval, an ellipsoidal, an oblong, a semi-circular, a triangular, a square, a rectangular, a pentagonal, a hexagonal, a heptagonal, an octagonal, a nonagonal, a decagonal, or a polygonal having more than ten sided, cross-section. In one or more embodiment(s), the suturing needle comprises multiple varied cross sections, including any combinations of the foregoing cross-sections.

Some of these suture needles can be used for medical purposes (e.g., suturing). For example, some of these suture needles can be used in transcatheter suturing, transcatheter intracardiac (or another body organ) suturing, and other flexible platform applications (e.g., endoscopic suturing, colonoscopic suturing). For example, rigid shaft embodiments, as commonly used for laparoscopic, video-assisted thoracoscopic surgery (VATS) and robotic surgery can be used to fundamentally transform suture-based tissue approximation/anchoring from a laborious task to a swift semi-automated or fully-automated endeavor. For example, some of these suture needles can be used for an open surgery, a minimally invasive surgery, a laparoscopic surgery, or an end effector robotic surgery. As such, the some of these suture needles can be used for manual surgery or automated surgery. Some examples of surgeries where some of these suture needles can be employed include laparoscopic surgery, robotic surgery, video-assisted or unassisted thoracoscopic surgery, arthroscopic surgery, natural orifice surgery, endoscopic surgery, gynecologic surgery, cardiac surgery, colorectal surgery, pulmonary surgery, gastric bypass surgery, hysterectomy surgery, dental surgery, urological surgery, brain surgery, fetal surgery or bariatric surgery, or among many others in human (e.g., between newborn until 120 years old, male, female) or animal (e.g., mammal, birds, fish, land animals) applications.

Note that some of these suture needles can be employed in medical or non-medical settings, whether on an object is animate or inanimate. For example, the object, when animate, can include a tissue, an organ, a body part, whether of human or animal, or others. For example, the tissue can be a muscle tissue, a bone tissue, a nerve tissue, an organ tissue, or others. For example, the object, when inanimate, can include a medical device, a prosthesis, an implantable, a machine, a surgical instrument, or others. For example, some of the non-medical setting can include garment making, fabric stitching, knot applications, sowing, shoe making, or others.

Current swaged suture needles generally include a lip or step-up in the transition from the suture thread to the thread attachment connector, and another lip or step-up in the transition from the thread attachment component to the needle body. Alone or in combination, these transitions pose a problem for the rollers of a rotational needle driver, as the suture thread may be damaged by the rollers, or conversely, the transitions may damage the rollers of the needle driver. For example, the rollers may roll past the thread attachment component of the needle and may clamp down on that region and damage/cut the suture thread.

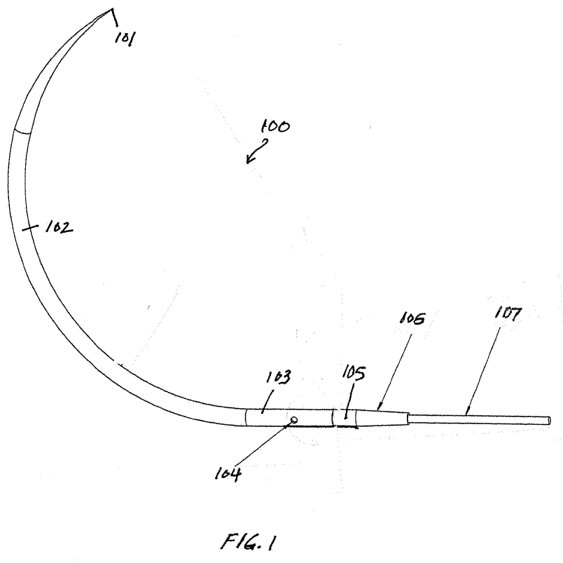

The present disclosure overcomes these deficiencies by incorporating an extension portion having a tapered portion that provides a smooth transition from the suture thread to the needle body. With reference to FIG. 1, a suture needle 100 of the present disclosure may comprise a tip portion 101 at proximal end of suture needle 100 for piercing a tissue, a needle body 102, the proximal end of which extends from the distal end of the needle tip 101, a thread attachment component 103 with a viewing port 104, the proximal end of which extends from the distal end of the needle body 102, an extension portion 105 comprising a tapered portion 106, the proximal end of the extension portion 105 extending from the distal end of the thread attachment component 103, and a suture thread 107. The suture thread 107 is swaged or crimped in the thread attachment component when the suture thread is visible through the viewing port 104. The needle tip portion 101 may be a tapered tip wherein the needle tip portion is round and tapers smoothly to a point, as shown in FIG. 1. Alternatively, the needle tip portion may be triangular, and may have a sharpened cutting edge on the inside or on the outside, or may have a “trocar point” or “tapercut” whereby the needle body is round and tapered, but ends in a small triangular cutting point. Alternatively, the needle tip may be a blunt point, such as for suturing friable tissues, or may have a needle tip portion that includes “side cutting” or “spatula points” whereby the needle is flat on top and bottom with a cutting edge along the front to one side (these are typically used for eye surgery). The needle body 102 is shown to have a curve in the drawings. Exemplary curves include at least half curved or ski, ¼ circle, ⅜ circle, ½ circle, such as shown in the figures, ⅝ circle, and compound curve. Alternatively, the needle body may be straight, such that the present disclosure may comprise a straight needle.

The length of extension portion 105 may be determined by the optimum balance between the minimal length needed by the surgeon to grab the needle by the “extension” when inserting it or withdrawing it to a surgical site, such as through a laparoscopic port, and the maximal length that can feasibly allow tapering to go through tissue following the needle path without causing tissue trauma. In certain embodiments, the tapered portion may comprise a magnetic material or a magentizable material. In certain embodiments, the suture material may comprise a magnetic material or a magnetizable material.

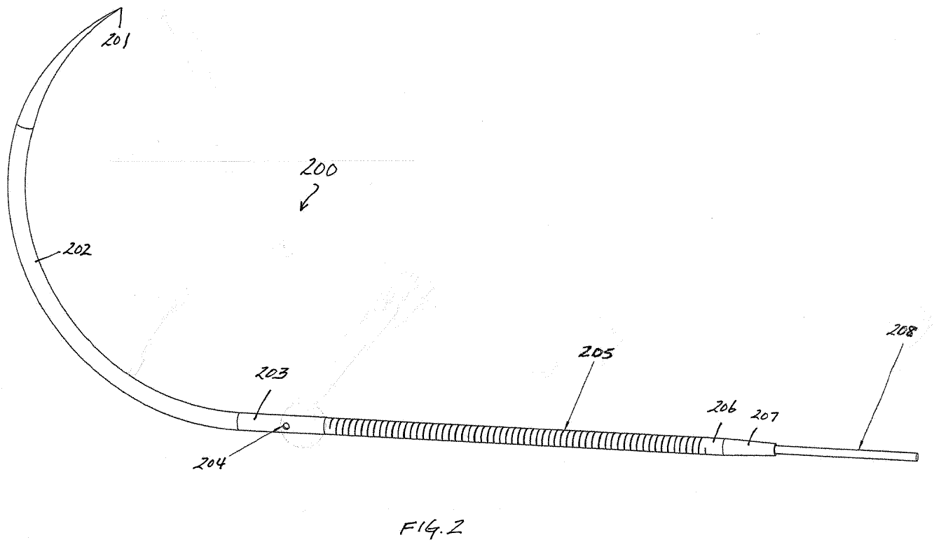

With reference to FIG. 2, another embodiment of a curved suturing needle 200 is shown. Curved suturing needle 200 comprises a tip portion 201 at proximal end of suture needle 200 for piecing a tissue, a needle body 202, the proximal end of which extends from the distal end of the needle tip 201, a thread attachment component 203 with a viewing port 204, the proximal end of which extends from the distal end of the needle body 202, a hollow flexible extension portion 205, the proximal end of which extends from the distal end of the thread attachment component 204, a ramp portion of the flexible extension portion 206 comprising a tapered portion 207, the proximal end of the ramp portion 206 extending from the distal end of the hollow flexible extension portion 205, and a suture thread 208. In the embodiment shown in FIG. 2 the hollow flexible extension portion 205 is created using laser cuts in a spiral or helical pattern. The suture thread 208 is swaged or crimped in the thread attachment component when the suture thread is visible through the viewing port 204.

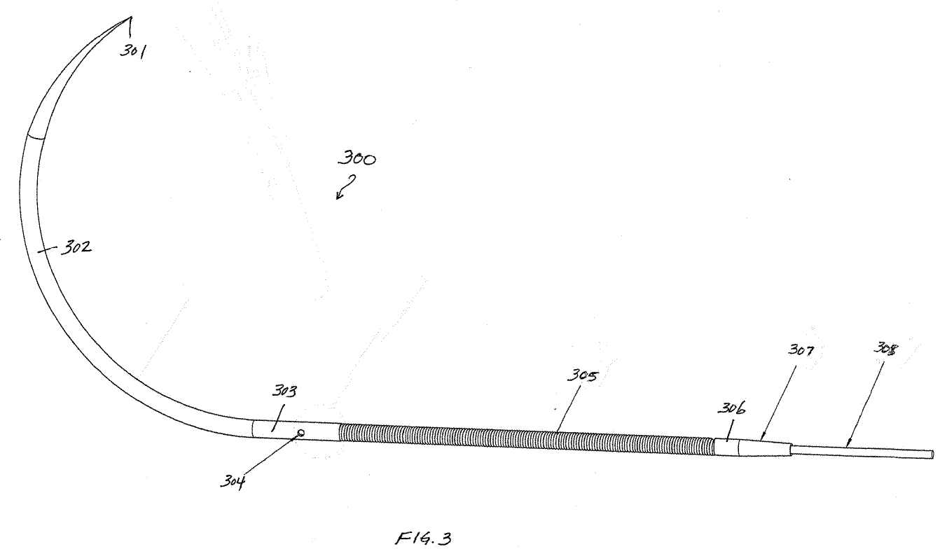

With reference to FIG. 3, another embodiment of a curved suturing needle 300 is shown. Curved suturing needle 300 comprises a tip portion 301 at proximal end of suture needle 300 for piecing a tissue, a needle body 302, the proximal end of which extends from the distal end of the needle tip 301, a thread attachment component 303 with a viewing port 304, the proximal end of which extends from the distal end of the needle body 302, a hollow flexible extension portion 305, the proximal end of which extends from the distal end of the thread attachment component 304, a ramp portion 306 of the hollow flexible extension portion 305 comprising a tapered portion 307, the proximal end of the ramp portion 306 extending from the distal end of the hollow flexible extension portion 305, and a suture thread 308. In the embodiment shown in FIG. 3 the hollow flexible extension portion 305 is created using wire shaped or formed into a spiral or helical pattern. The suture thread 308 is swaged or crimped in the thread attachment component when the suture thread is visible through the viewing port 304.

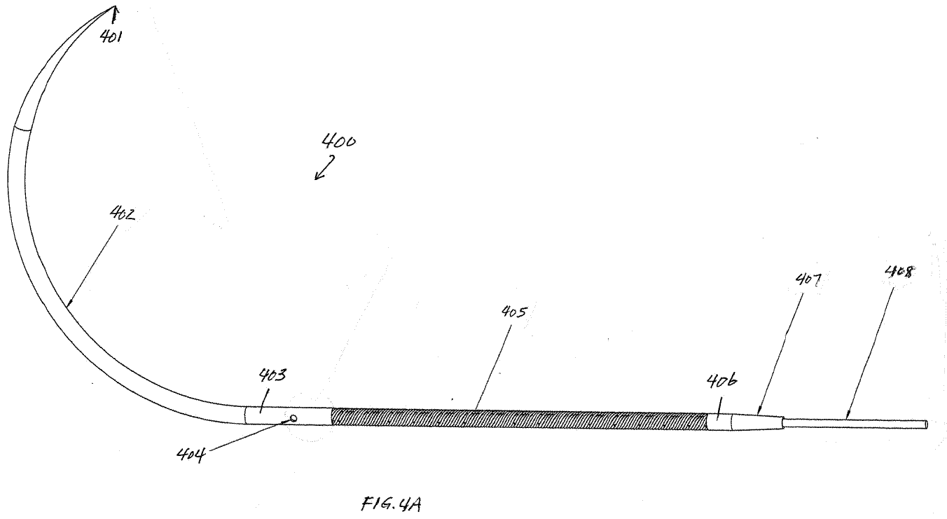

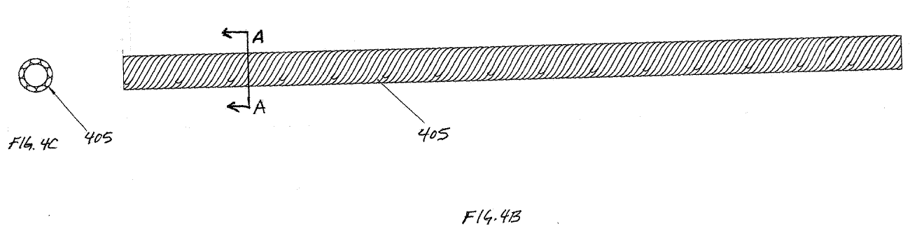

With reference to FIG. 4A, another embodiment of a curved suturing needle 400 is shown. Curved suturing needle 400 comprises a tip portion 401 at proximal end of suture needle 400 for piecing a tissue, a needle body 402, the proximal end of which extends from the distal end of the needle tip 401, a thread attachment component 403 with a viewing port 404, the proximal end of which extends from the distal end of the needle body 402, a hollow flexible extension portion 405, the proximal end of which extends from the distal end of the thread attachment component 404, a ramp portion of the hollow flexible extension portion 406 comprising a tapered portion 407, the proximal end of the ramp portion 406 extending from the distal end of the hollow flexible extension portion 405, and a suture thread 408. In the embodiment shown in FIG. 4 the hollow flexible extension portion 405 is created using wire cable shaped into a spiral or helical pattern. The suture thread 408 is swaged or crimped in the thread attachment component when the suture thread is visible through the viewing port 404.

With reference to FIG. 4B, the wire cable hollow flexible extension portion 405 from FIG. 4A is shown. FIG. 4C shows a cross-section of the wire cable hollow flexible extension portion 405 from FIG. 4B along line A-A.

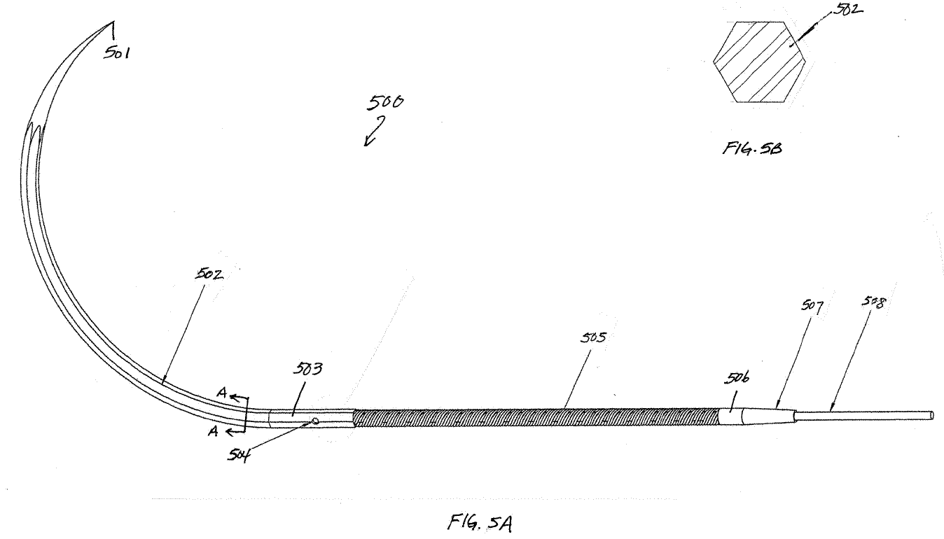

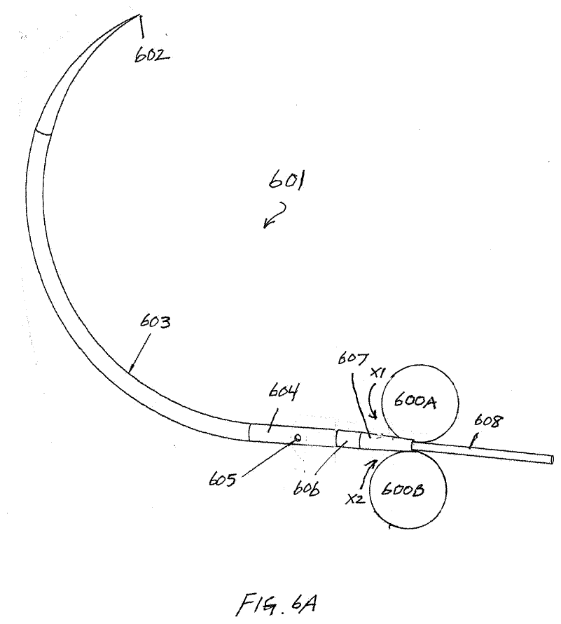

With reference to FIG. 5A, another embodiment of a curved suturing needle 500 is shown. Curved suturing needle 500 has a needle tip portion 501 at proximal end of suture needle 500 for piercing a tissue, a non-smooth hexagonal needle body 502, and thread attachment component 503 with a viewing port 504. The proximal end of the non-smooth needle body 502 extends from the distal end of the needle tip 501, and the proximal end of the thread attachment component 503 extends from the distal end of the needle body 502. Curved suturing needle 500 also comprises a hollow flexible extension portion 505, the proximal end of which extends from the distal end of the thread attachment component 503, a ramp portion of the hollow flexible extension portion 506 comprising a tapered portion 507, the proximal end of the ramp portion 506 extending from the distal end of the hollow flexible extension portion 505, and a suture thread 508. In the embodiment shown in FIG. 5A the hollow flexible extension portion 505 is created using wire cable shaped or formed into a spiral or helical pattern. The suture thread 508 is swaged or crimped in the thread attachment component when the suture thread is visible through the viewing port 504. FIG. 5B shows a cross-section of the needle body 502 from FIG. 5A along line A-A.

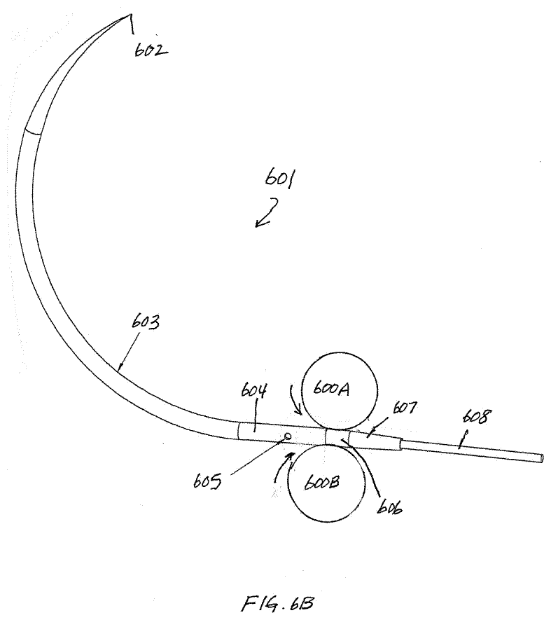

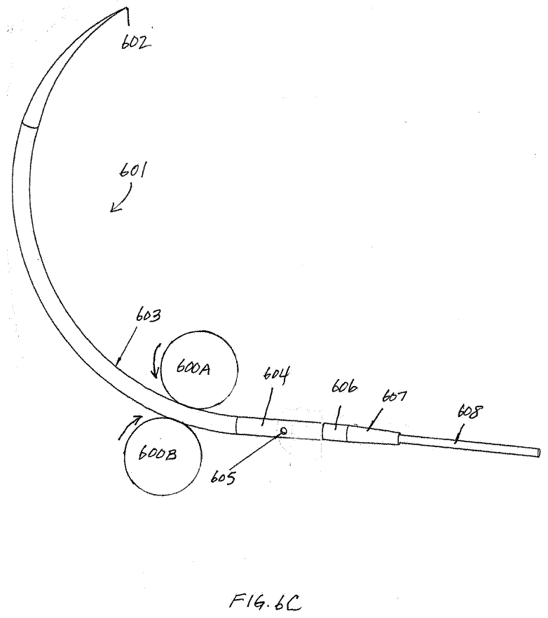

These and other embodiments of the curved suturing needle create a procedural benefit and efficiency by loading the needle onto the needle driver to a position ready for suturing all in a single step, as shown in FIGS. 6A-6C. The rollers 600A and 600B may be closed over the suture material 608 and may rotate in opposite directions (x1 and x2, respectively) to advance the needle forward, shown as the transition from the needle position in FIG. 6A to FIG. 6C. As the needle advances between the rollers of the needle driver, the rollers smoothly advance the needle from the suture material to the tapered portion 607 of the ramp portion 606 (FIG. 6A). Without opening the rollers, the rollers may continue to rotate to advance the needle along the tapered portion 607 over the ramp portion 606 (FIG. 2B).

In the absence of the tapered portion 607, this would represent a step or lip on the needle that may be damaged by the rollers, or conversely, that may damage the rollers. Accordingly, in the absence of the tapered portion 607, the clinician would have to use a grasper to hold/stabilize the needle, then open the needle driver to release the suture it was grasping during needle insertion and then grasp the needle with the needle driver once positioned over the needle body in preparation for suturing. In a laparoscopic setting each of these steps may require time, effort, coordination and maybe even repetition due to the lack of depth perception (2visualization systems used mostly) and decreased dexterity, fulcrum effect of port, shape, and design. Finally, as shown in FIG. 6C, the rotation of the rollers advances the needle so that it is positioned between the rollers and properly loaded for suturing without ever opening the rollers. These motions may be reversed to unload the needle, such as to remove the needle from a suture site (e.g., from the abdomen or from a laparoscopic surgical site).

The material configuring the suture needle is not limited, and may comprise a metal or a metal alloy, such as, for example, a biocompatible metal or metal alloy. In certain embodiments, the suture needle comprises any of steel wire, a martensitic stainless steel, or an austenitic stainless steel. In certain embodiments, the suture needle is comprised of a magnetic or magnetizable material. When formed of steel wire or martensitic stainless steel, the needle may be hardened by thermal treatment. Other processes when forming or finishing the needle may be siliconization, coating with any number or combination of biocompatible coating materials or lubrication with any number or combination of biocompatible lubricating agents, among other processes.

As shown in FIGS. 1-6C, for example, the suture thread may be swaged to the needle by the thread attachment component. Accordingly, the needle is generally an atraumatic needle, i.e., eyeless needle, having a suture material or thread attached at an end by swaging whereby the suture material is inserted into a channel at the blunt end of the needle, such as into the thread attachment component, which is then deformed to a final shape to hold the suture and needle together. The needle may be permanently swaged to the suture material or may be designed to come off the suture material with a sharp straight tug. These “pop-offs” are commonly used for interrupted sutures, where each suture is only passed once and then tied.

The suture material or thread may be mono-filamentary, i.e., formed of a single filament, or multi-filamentary, i.e., formed from a combination of two or more filaments, e.g., three filaments arranged in a braided fashion [symbol] formed from a combination of two or more filaments, e.g., three filaments arranged in a braided/twisted fashion within a smooth outer sleeve of extruded material. The suture thread has a length, where that length is typically at least 5 inches, or at least 10 inches, or at least 15 inches, or at least 20 inches. The suture thread will typically have two ends, which may be described as a deployment end and/or a trailing end. In such a case, the deployment end of the suture thread is that end that first enters tissue, adjacent to the needle, such as connected via the thread attachment component to the distal end of the needle body of the needle. Alternatively, the suture material may be looped, such that each of the two free ends are connected to the needle by the thread attachment component.

The thread can be a suture, which can be non-absorbable or absorbable of various gauges. The thread can include silk, cotton, fabric, nylon, polyester, silver, copper, Dacron, rubber, silicon, plain or chromic catgut, polyglycolide, polydioxanone, monocryl, polypropylene, triclosan, caprolactone, polymer, glycolide, l-lactide, p-dioxanone, trimethylene carbonate, ε-caprolactone, stainless steel, ceramic, glass, leather, or other natural or artificial materials. The suture thread may comprise metallic wire suture (as used for sternal closure during open heart surgery) or any other biocompatible material used for approximating human or animal tissues. The thread can also comprise any material used to stitch or otherwise approximate, or otherwise connect in any way, inanimate objects of any sort. The thread is solid, but can be perforated. The thread is internally dense, but can be hollow. The thread can be rigid, semi-rigid, elastic, resilient, or flexible. For example, the thread can bend about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). The thread can have a cross-section that is closed-shaped (e.g., O-shape, D-shape, O-shape, square, rectangle, triangle, polygon) or open-shaped (e.g., U-shape, C-shape, V-shape), whether symmetrical or asymmetrical.

The suture material or thread may be bioabsorbable, such that after introduction into a tissue it is broken down and absorbed by the body. Typically, the degradation process is at least partially mediated by, or performed in, a biological system. Accordingly, bioabsorbable refers to a chain scission process by which a polymer chain is cleaved through various mechanisms, including, for example, by chemical reaction (e.g., hydrolysis, oxidation/reduction, enzymatic mechanisms or a combination of these) or by a thermal or photolytic process. Bioabsorbable suture material may include polymers such as polyglycolic acid, copolymers of glycolide and lactide, copolymers of trimethylene carbonate and glycolide with diethylene glycol (e.g., MAXON™, Tyco Healthcare Group), terpolymer composed of glycolide, trimethylene carbonate, and dioxanone (e.g., BIOSYN™ [glycolide (60%), trimethylene carbonate (26%), and dioxanone (14%)], Tyco Healthcare Group), copolymers of glycolide, caprolactone, trimethylene carbonate, and lactide (e.g., CAPROSYN™, Tyco Healthcare Group). In certain embodiments, the bioabsorbable suture material may comprise or include any other polymer useful for suturing applications that currently exists or that may be developed in the future.

Alternatively, the suture material or thread may be non-degradable, such that it is not degraded by chemical, thermal, or photolytic process. Non-degradable suture material includes polyamide (also known as nylon, such as nylon 6 and nylon 6.6), polyester (e.g., polyethylene terephthlate), polytetrafluoroethylene (e.g., expanded polytetrafluoroethylene), polyether-ester such as polybutester (block copolymer of butylene terephthalate and polytetra methylene ether glycol), polyurethane, metal alloys, metal (e.g., stainless steel wire), polypropylene, polyethelene, silk, and cotton. Sutures made of non-degradable suture material are particularly suitable for applications in which the suture is meant to remain permanently or is meant to be physically removed from the body. In certain embodiments, the non-degradable suture material may comprise or include any other polymer useful for suturing applications that currently exists or that may be developed in the future.

The suture material or thread may comprise a coating or agent applied to a surface thereof that may affect wound healing, such as a coating material, wound healing agent, antimicrobial agent, antibacterial agent, growth factor, adhesive, sealant, blood product, blood component, preservative, anti-adhesive, protein, polysaccharide, peptide, genetic material, viral vector, nucleic acid, nucleotide, plasmid, lymphokine, radioactive agent, metal, alloy, salt, growth factor, growth factor antagonist, cell, hydrophobic agent, hydrophilic agent, immunological agent, anti-colonization agent, and combinations thereof. The suture material or thread may comprise a coating or agent applied to a surface thereof that may enhance the surgeon's ability to accurately suture, such as colorants, dyes, ultraviolet absorbers, ultraviolet stabilizers, photochromic agents, diagnostic agent, imaging agent, radiopaque agent, or combinations thereof. The suture material or thread may comprise a coating or agent applied to a surface thereof that may reduce the friction or drag of the suture material or thread as it is passes through tissue.

The suture material or thread may have a diameter indicated by a cross-sectional dimension. This diameter may be determined at a location along the suture where there are either no barbs, or the barbs that are present are pushed against the suture body so that they are flush with the surface of the suture body. The suture may have no barbs or may have barbs along all or only a portion of the suture length. According to certain embodiments, the suture may have at least one barbed region positioned distal from the thread attachment component. Such positioning may allow the presently disclosed suture needle to be backed out of a suture site, wherein the suture material adjacent the needle would not have barbs and would easily slide back through the tissue. In some embodiments the tapered portion covers the barbs on the suture, thereby allowing the suture needle to be backed out of the suture sight. More distally located barbed suture may remain securely in place once positioned in the tissue.

The suture may have a generally circular cross-sectional shape, or may have a non-circular shape, e.g., polygonal such as 3-sided (triangular), or 4-, 5- or 6-sided (hexagonal) sided or a shape described by a polygon with any number of sides. The cross section of the suture body may have an oval, an ellipsoid, an oblong, or a semi-circular appearance.

Suture sizing is based upon diameter. The United States Pharmacopeia (“USP”) designation of suture size runs from 0 to 7 in the larger range and 1-0 to 11-0 in the smaller range; in the smaller range, the higher the value preceding the hyphenated zero, the smaller the suture diameter. Under the USP nomenclature system, the actual diameter of a suture will depend on the suture material, so that, by way of example, a suture of size 5-0 and made of collagen will have a diameter of 0.15 mm, while sutures having the same USP size designation but made of a synthetic absorbable material or a non-absorbable material will each have a diameter of 0.1 mm. The selection of suture size for a particular purpose depends upon factors such as the nature of the tissue to be sutured and the importance of cosmetic concerns; while smaller sutures may be more easily manipulated through tight surgical sites and are associated with less scarring, the tensile strength of a suture manufactured from a given material tends to decrease with decreasing size. It is to be understood that the suture materials for use with the suture needles disclosed herein include without limitation 7, 6, 5, 4, 3, 2, 1, 0, 1-0, 2-0, 3-0, 4-0, 5-0, 6-0, 7-0, 8-0, 9-0, 10-0 and 11-0. It is to be understood that a variety of suture lengths may be used with the suture needles described herein.

The needle body may have any profile known in the art, such as circular, oval, triangular, and the like. The needle body may have a smooth surface. According to certain embodiments, the needle body may be a non-smooth profile. For example, when trying to drive needle through a tissue using a rotational needle drive, a smooth surface of the needle body may allow the needle to deviate out of an initial chosen plane of rotation, especially for needle bodies having an oval shape. Moreover, it is not possible to securely position these needles in a non-standard angle. Accordingly, an object of the present disclosure is to provide suture needles that may perform optimally in roller needle drivers and may incorporate features that enable multi-planar needle driving.

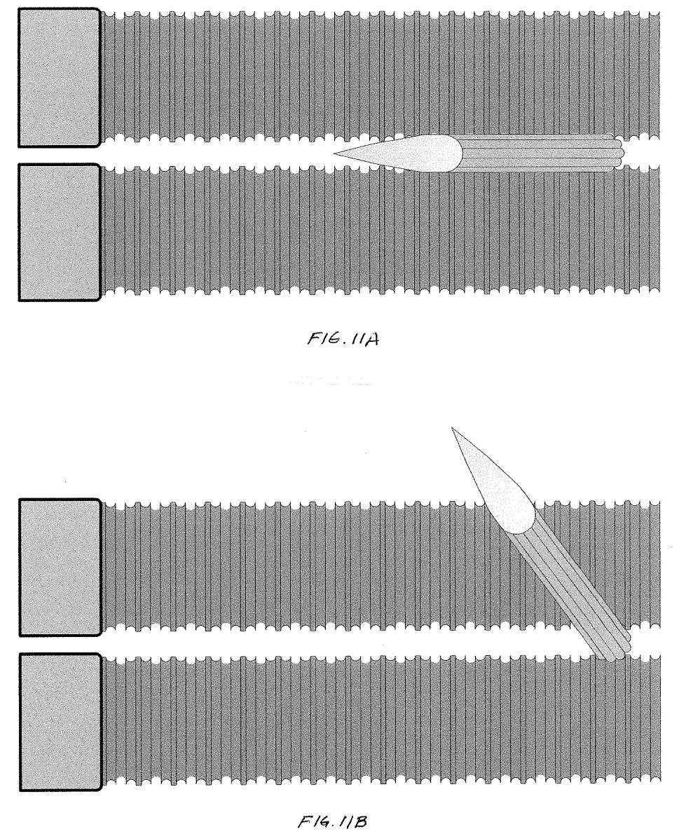

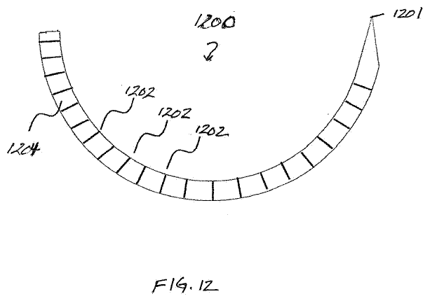

Rotational needle drivers generally comprise an actuator portion having linear and rotational motion systems and an interactive portion configured to interact with the suture needle. Each interactive portion comprises a first extended member and a second extended member which approximate a pair of rod-like scissors that may be opened and closed. Each extended member comprises a main needle-grasping portion having grooves, such as grooves 620 on each of a first 600A and second 600B roller as shown in FIG. 10A. The suture needles of the present disclosure are designed to include ridges that mate with these grooves 620 of the rollers so that a secure connection between the needle and the rollers may be achieved.

As shown in FIGS. 10A and 10B, the needle may be grasped between the rollers to position the needle perpendicular to the rollers, passing behind the rollers of the driver as in FIG. 10A or in front of the rollers as in FIG. 10B. The unique arrangement of ridges on the suture needle further provide for grasping between the two rollers at non-standard angles, such as parallel with the rollers as shown in FIG. 11A or even oblique to the rollers as shown in FIG. 11B, or any of a number of angles therebetween.

When the suturing procedure begins, the suture needle having a non-smooth surface configured to interact with the grooves of the two rollers is grasped at a desired angle. The surgeon may press an actuator on the needle driver that brings the rollers closer so that they may grasp the needle and may lock the rollers in position exerting the necessary force to maintain the needle still. Once the needle is tightened, the actuator may create a rotational movement of the rollers, such as shown in FIG. 6A, wherein the rollers rotate in directions opposite from each other to affect movement of the needle in either of two directions. Thus, the surgeon may drive the needle through the tissue as the rollers rotate. When the desired rotation is completed, the surgeon releases the locking mechanism. This method is repeated on the other tissue that is to be joined to the tissue already perforated by the needle, and each step above is repeated as many times as stitches have to be performed.

The design of the roller needle driver permits rotation of a curved suture needle without requiring a rotational motion of the wrist. Therefore, the instrument can be held in a position that may not allow wrist rotation and still carry out its function. In some situations, the structures to be sutured lie very deep. Doing surgery on the vertebral column of very obese patients is one such situation. For example, if the dura mater (a membrane that protects and envelops the spinal cord) is cut it needs to be sutured. The dura mater lies within a deep constricted space. In a very obese patient, the additional thickness of the adipose (fatty) tissue makes the dura lie even deeper from the surface. The surgeon may have to lean towards the patient and rotate the arm bearing the needle driver. This is necessary to get his forearm in a vertical position so he can maneuver the instrument properly. The needle driver used with a suture needle as disclosed herein may be operated in such a situation.

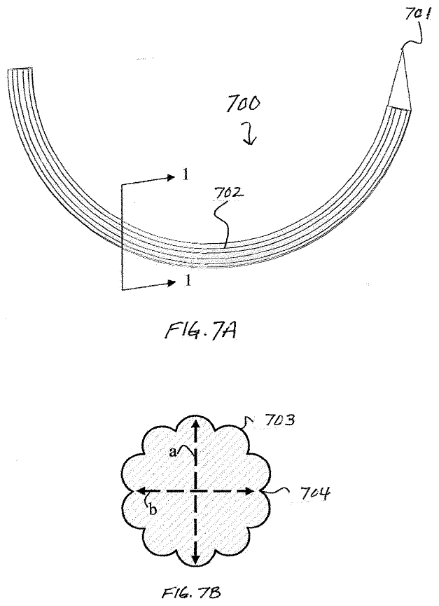

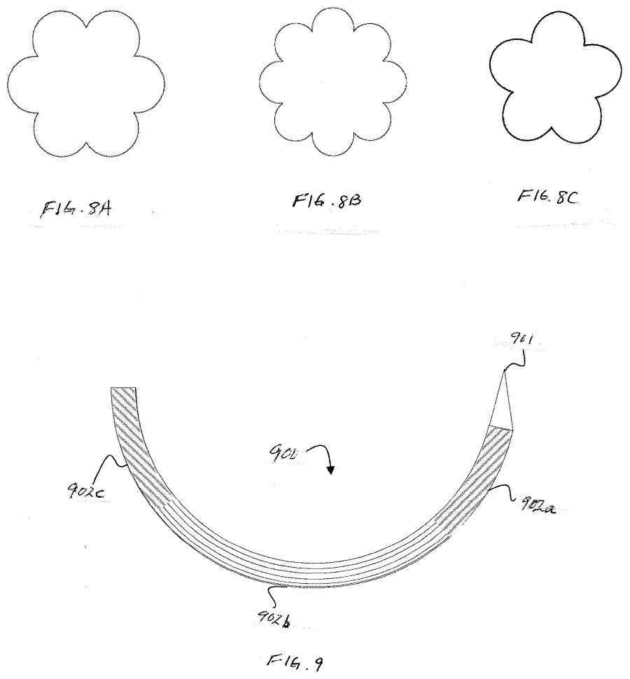

According to certain embodiments of the present disclosure, the needle 700 may comprise a non-smooth surface. With reference to FIG. 7B, a cross-sectional view of the curved needle body taken along line 1-1 of FIG. 7A shows this region to have a circular shape with a non-smooth profile. This non-smooth profile can be achieved by forming indentations 704 in the outer circumferential periphery of the needle body. As shown, these indentations may be evenly spaced, and may form protrusions 703, such as the smooth convex protrusions shown in FIG. 7B.

These indentations 704 and/or protrusions 703 create longitudinal ridges along the length of the needle body 702 and may be uniquely designed and configured to fit within the grooves of the rollers of a rotational needle driver. That is, these ridges may fit within the grooves of the rollers of the needle driver and act like rails to secure the orientation of the needle in the groove while the needle is being driven by the rotation of the rollers. Because the needle 700 has ridges evenly spaced about its round cross-section, the needle can be placed in any of a number of angles on the rollers. See for example the rollers (600A, 600B) shown in FIGS. 10A-11B that are grasping a needle along the needle body, wherein the ridges (indentations 704 and protrusions 703) match the grooves 620 of the plurality of rollers independent of the orientation of the needle. In certain embodiments, the ridges are unevenly spaced. In certain embodiments, the ridges have different heights. In certain embodiments, the ridges have different orientations.

With reference to FIG. 7B, the depth of the indentations 704 (difference in diameter a and b of the needle body 702), and their spacing about the circumference of the needle body defines the ridges. These dimensions may be selected so that the ridges match the depth and spacing of grooves on the rollers of a rotational needle driver (such as shown in FIGS. 10A-11B). The specific design shown in FIG. 7B is exemplary only, as many other designs and configurations are possible, such as shown in FIGS. 8A-8C which include different numbers of indentations and thus different numbers of convex protrusions (ridges). Moreover, while the protrusions or ridges are shown to have a generally convex shape without any sharp angles (i.e., no pointed edges), other configurations (such as hexagonal as shown in FIG. 5A) are possible and within the scope of the present disclosure.

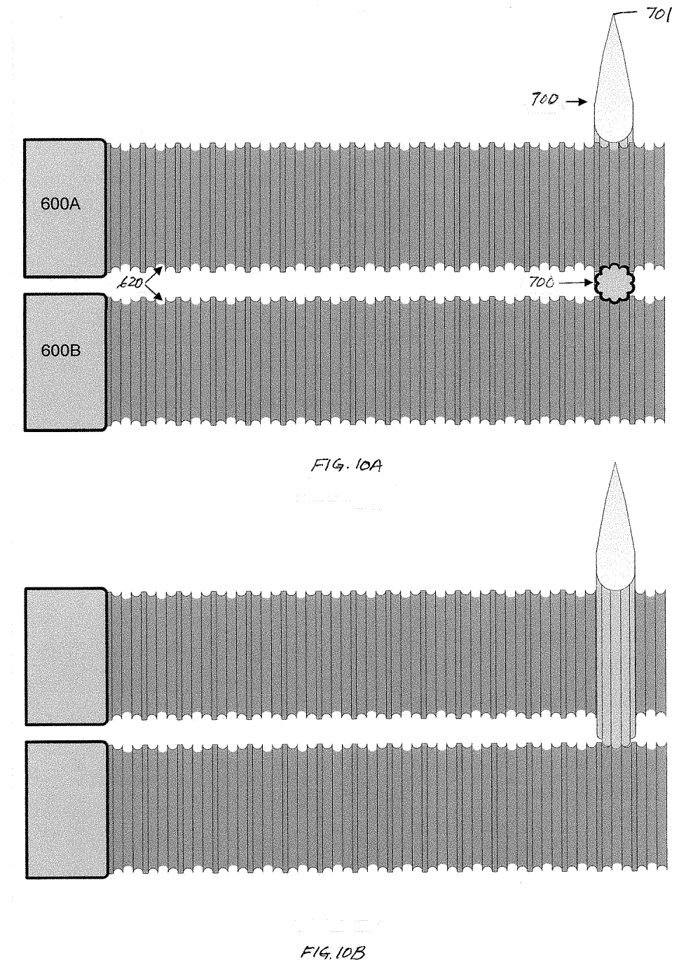

The ridges formed on the suture needle generally extend along a full longitudinal length of the needle body 702, as shown in FIG. 7A. According to certain embodiments, the indentations 704 may be formed about a circumference of the needle body at an angle, thus forming ridges (i.e., protrusions 703) that may spiral about the outer circumference of the needle body 702 of the needle 700. The angle may be selected so that the ridges have a right-handed or left-handed spiral. Moreover, as shown in FIG. 9, the angle of the ridges may be changed one or more times along a length of the needle body 902. For example, the ridges may have a right-handed angle (902c), no angle (902b), or a left-handed angle (902a). The specific angle, size of the angled region, and placement thereof may be selected based on suture procedure requirements.

In certain embodiments, for example, as shown in FIG. 12, the suture needle 1200 includes needle tip portion 1201 and needle body 1204 including indentations and/or ridges 1202 that extend circumferentially around the suture needle body, such that they provide improved traction for the rollers of the needle driver to move the suture needle through dense, compact, or hard tissue material. In certain embodiments, the circumferential ridges 1202 may be closely spaced one from the other. In certain embodiments, the circumferential ridges 1202 may be spaced apart one from the other. In certain embodiments, the distance between the circumferential ridges 1202 is fixed. In certain embodiments, the distance between circumferential ridges 1202 varies from ridge to ridge. In certain embodiments, the distance between circumferential ridges 1202 varies according to a predetermined pattern, formula, or algorithm. In certain embodiments, one or more of the rollers of the needle driver include indentations or ridges that run from a proximal end to a distal end of one or more of the grooves of the rollers. In certain of the embodiments, the indentations or ridges in the grooves of the rollers mate or interleave with the circumferential ridges 1202 or indentations on the suture needle. This provides the rollers with improved traction for the rollers of the needle driver to move the suture needle through dense, compact, or hard tissue material or inanimate material.

In certain embodiments, the suture needle includes ridges that extend along only a portion of the needle trunk. In certain embodiments, the suture needle includes ridges that begin at a first portion and end at a second portion of the needle trunk. In certain embodiments, the suture needle includes multiple discontinuous areas having ridges with intervening non-ridged areas. In certain embodiments, the suture needle includes certain areas with at least one of ridges, a groove surface, a roughened surface, a smooth surface, a bumpy surface, a ribbed surface, a tacky surface, or a polished surface. In certain embodiments, the suture needle includes certain areas with at least one of a ridged surface, a grooved surface, a roughened surface, a smooth surface, a bumpy surface, a ribbed surface, a tacky surface, or a polished surface, and other areas with at least one different surface. In certain embodiments, the ridged and non-ridged sections, or any combinations thereof may each be of any particular length, width, order, or orientation. In certain embodiments, the ridged surface, the grooved surface, the roughened surface, the smooth surface, the bumpy surface, the ribbed surface, the tacky surface, or the polished surface sections, or any combinations thereof may each be of any particular length, width, order, or orientation. In certain embodiments, there may be more than one type of non-ridged surface, such as, for example, a ridged surface, a grooved surface, a roughened surface, a smooth surface, a bumpy surface, a ribbed surface, a tacky surface, or a polished surface. For example, ridged sections, smooth sections and otherwise textured non-ridged sections can be combined in the same needle trunk for any type of application.

In certain embodiments, a suture needle may comprise a smooth tip section, ridged body and textured-non-ridged portion near the swage end that could induce some slight vibration captured by a robotic needle driver to improve haptic feedback to the surgeon on when to pull the needle out of the tissue to minimize tissue trauma, particularly in regions where tissue visibility or maneuverability is quite limited or tissues are very prone to tearing if the needle is pulled from the tissues too soon.

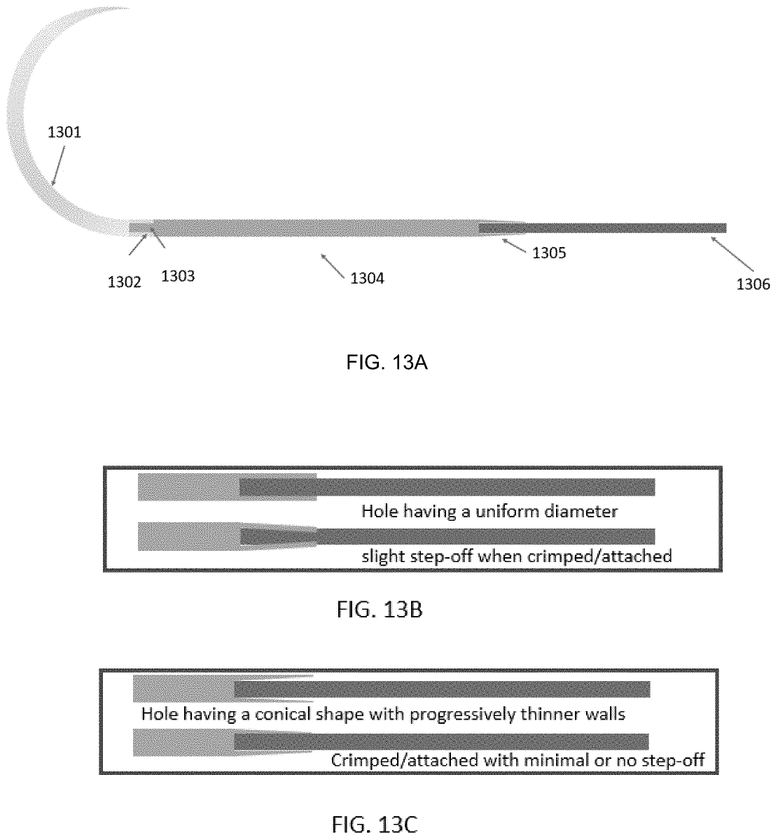

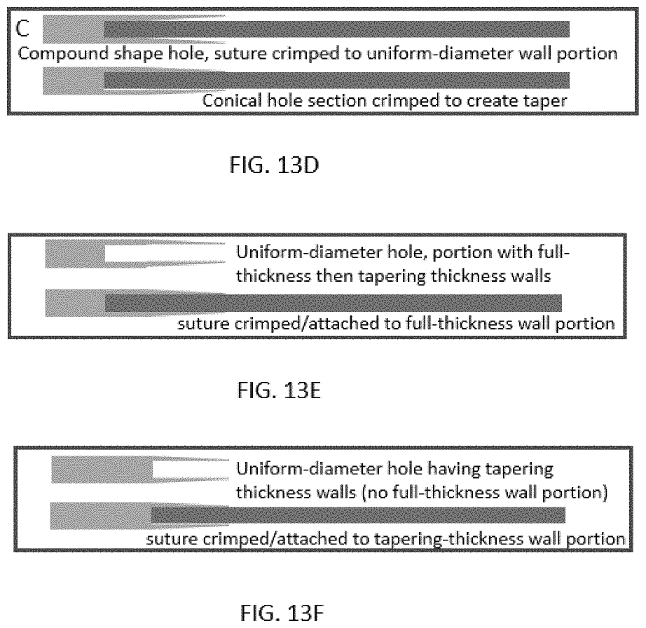

With reference to FIG. 13A, another embodiment of a curved suturing needle 1301 is shown. Curved suturing needle 1301 comprises an extension attachment portion 1302 for crimping or otherwise attaching single-piece Nitinol (or any other material) extension, a portion 1303 of the extension with a smaller diameter to facilitate crimping (or otherwise attaching) the extension to the needle, a body 1304 of the single-piece nitinol extension having the same diameter as wire diameter of the needle, a suture-connecting/attachment portion 1305 of the single-piece nitinol extension having a tapered configuration, and suture thread 1306. In the embodiment shown in FIG. 13A the single-piece extension is shown, which can be made out of nitinol, any other suitable metal or metal alloy or any other suitable material. This single-piece extension has a decreased diameter at its distal end for being crimped to (or otherwise attached in any suitable way) to the needle. The rest of the body of the extension has a wire diameter that equals the wire diameter of the needle. The proximal (trailing) end of the extension serves as an attachment portion to crimp (or otherwise attach in any suitable way) a suture thread. This can be accomplished by drilling (laser-drilling, mechanical drilling or otherwise) or otherwise forming a hole at the proximal part of the single-piece extension for suture attachment. Although the exemplary embodiment shown here depicts a straight (linear) configuration for the extension, any and all non-straight (non-linear) extension iterations such as curved, J-shaped, S-shaped, L-shaped, compound curved, angled, multi-angled, spiral, corkscrew/helical or any other non-linear shaped extension can be used in any of the needle extensions disclosed herein. The non-linear extension configurations are meant to apply to all single-piece extension embodiments disclosed herein as well as all multi-component extension embodiments and different combinations and permutations of these configurations.

All the embodiments describing needles with smooth and non-smooth surfaces as well as all the variations for attachment of an extension to a needle (including a straight needle) and all the embodiments describing different combinations of needle configuration and suture material (barbed, non-barbed, monofilamentous, braided, absorbable, non-absorbable, or as otherwise described herein) are applicable to the single-piece nitinol extension. The cross-section of the single-piece extension and the cross-section of the needle can each be (independent of each other) round, oval, square, pentagonal, hexagonal or any other shape as disclosed herein. All of the variations for connecting the needle to the extension or the extension to the suture thread apply to all the single-piece extension embodiments disclosed herein.

With reference to FIG. 13B, FIG. 13C, FIG. 13D, FIG. 13E and FIG. 13F, shown are multiple iterations describing how to connect the single-piece nitinol extension. Iterations describing the connection between the single-piece extension and the suture thread (applicable to all embodiments disclosed herein). The hole can have a uniform diameter which produces a slight step-off when crimped or attached (FIG. 13B) or be conical (FIG. 13C), having a progressively larger diameter towards the proximal end of the single-piece extension as to create progressively thinner walls. The purpose of this conical hole is to minimize the step-off created by the wall thickness of this crimping section to create the smoothest conical taper possible between the diameter of the single-piece extension and the diameter of the suture. Another variation of this tapered suture connection includes having the hole have a compound shape (FIG. 13D), with a uniform-diameter portion and a conical shape portion (to facilitate suture thread insertion). In this variation, the suture could be crimped (or otherwise attached) to the uniform-diameter portion and have the conical portion crimped (or mechanically or otherwise deformed) to reduce the outer diameter at the suture-connecting portion of the single-piece extension and create the smoothest taper possible. Another iteration comprises the suture-connecting component to have a hole of a uniform diameter with the walls having a portion that is full-thickness then becoming progressively thinner as to create the desired tapered configuration (FIG. 13E). In this iteration, the suture could be crimped (or otherwise coupled or attached) to the section of the hole having full-thickness walls. In another iteration, the walls of the hole become progressively thinner (no full-thickness portion) and the suture is crimped (or otherwise coupled or attached) to that section of the extension (FIG. 13F). These iterations apply to any needle disclosed herein but may be more practical for smaller wire diameter needles (including, but not limited to, RB-1 and others). All these iterations described herein to connect the extension and suture thread are to be applied to how the needle is connected to the extension, may that be a single-piece extension or any other iteration of the extension, made of nitinol, any other suitable material or any combination of materials and components thereof.

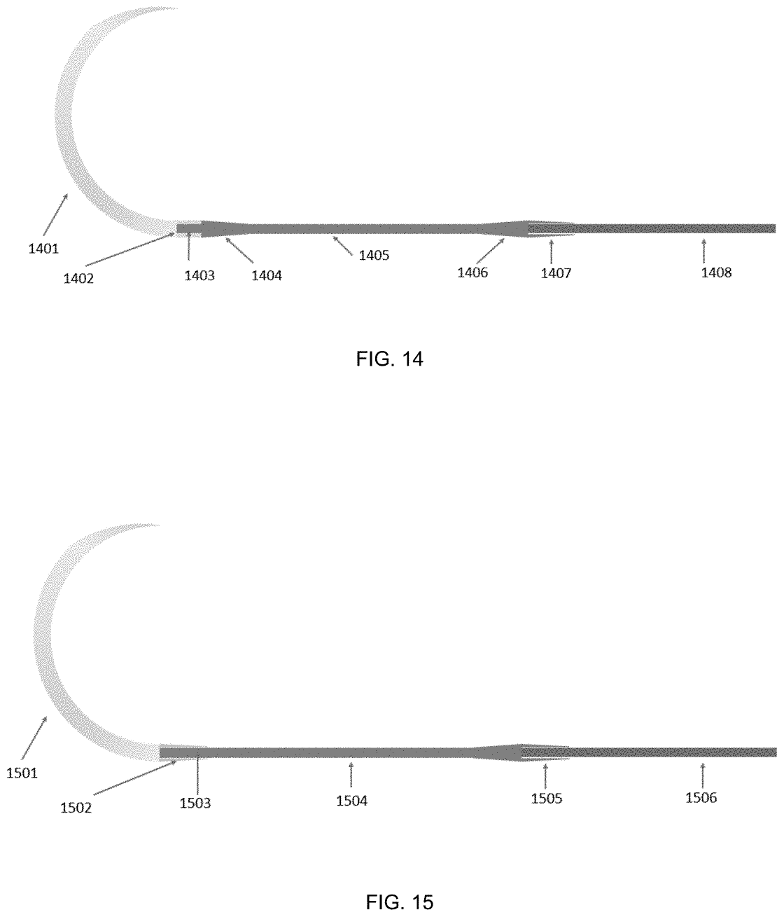

With reference to FIG. 14, another embodiment of a curved suturing needle 1401 is shown. Curved suturing needle 1401 comprises an extension attachment portion 1402 for crimping or otherwise attaching single-piece Nitinol (or any other material) extension, a reduced diameter portion 1403 at the distal end of extension, a tapered portion 1404 of distal end of body of extension 1405, a tapered portion 1406 of proximal end of body of extension, a suture-attachment portion 1407 of the extension, and a suture thread 1408. This embodiment shows a single-piece nitinol (or any other metal, metal alloy or suitable material) extension 1405 that has a distal end (closest to the needle) having a diameter smaller than the diameter of the needle 1401 (for the extension to be crimped or otherwise attached) to the needle, followed by a portion 1404 that tapers down in diameter from a larger diameter of the needle to a smaller diameter comprising a length of the single-piece extension 1405. This length of the extension is followed by an area in which the diameter of the extension tapers up (1406), gradually increasing in diameter from the smaller diameter of the extension body to the diameter of the needle. In turn, this area of the extension is followed by a suture attachment portion 1407 that tapers down to approximate the diameter of the suture thread 1408. All the multiple iterations disclosed herein for connecting the single-piece extension to the suture can also be applied to this embodiment. The rationale for this embodiment is that a wire with a thinner diameter may be more flexible than a thicker wire made of the same material. For larger needles having thicker wire diameters (SH needle, for example), creating a flexible single-piece nitinol extension that matches the wire diameter of the needle (to create a seamless transition between the needle and the extension) becomes progressively more difficult as the size of the needle increases. A solution is described in this embodiment. The body of the extension has a wire diameter that is thinner than that of the needle. Still, in order to create a smooth transition between the extension and the needle, the extension includes an area in which its diameter gradually increases until it matches the wire diameter of the needle. This area of the extension is contiguous with the proximal end of the needle, eliminating any step-off between the needle and the extension. The proximal end of the body of the extension (the end closest to the suture attachment portion) also gradually increases in diameter to ultimately match the wire diameter of the needle. This is done to accommodate suture thread sizes that usually accompany any given needle that has a wire diameter larger than the wire diameter of the body of the extension. A suture attachment portion follows the body of the extension. This suture attachment portion of the extension is tapered and any of the iterations for creating this tapered suture connection portion (FIG. 13B-F, and all their combinations thereof) can also be used to describe this portion of the extension in this and all other embodiments disclosed herein.

With reference to FIG. 15, another embodiment of a curved suturing needle 1501 is shown. Curved suturing needle 1501 comprises a tapered extension attachment portion 1502 for crimping or otherwise attaching single-piece Nitinol (or any other material) extension, a distal end of extension 1503 with wire diameter equal to the body of the extension, the body of extension 1504, a suture-attachment portion 1505 of the extension, and a suture thread 1506. In this iteration, the portion of the needle to which the single-piece extension (or any type of extension disclosed herein) is crimped (or otherwise attached to) is tapered as to gradually decrease in diameter from the wire diameter of the needle to approximate the wire diameter of the extension, leaving only a very small step-off between the needle and the extension. This tapered extension-attachment portion of the needle and its connection to the extension can take the form of any given number of iterations disclosed herein (equally applicable for both the connection between the extension and the suture and the connection between the needle and the extension). In this iteration, the distal end of the extension (the end connecting to the needle) has a uniform wire diameter equal to the wire diameter of the body of the extension (no tapering or bulging). The proximal end of the extension (the trailing end connecting to the suture) and the portion of the extension connecting to the suture are described in the same fashion as in FIG. 14. All the iterations disclosed previously to describe the connection between the extension and the suture in FIG. 13 and FIG. 14 also apply to describe the suture connection portion of the present embodiment.

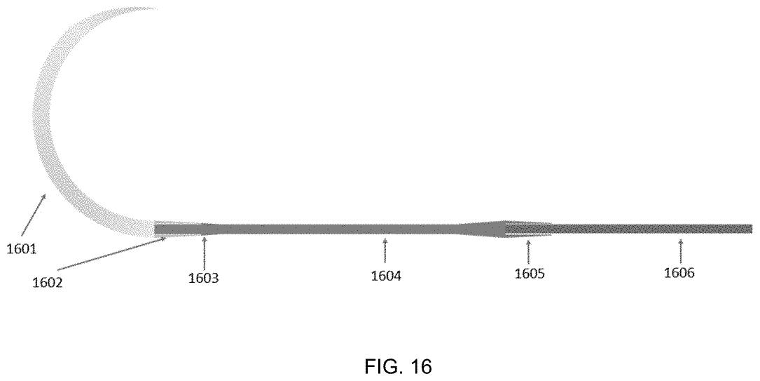

With reference to FIG. 16, another embodiment of a curved suturing needle 1601 is shown. Curved suturing needle 1601 comprises a tapered extension attachment portion 1602 for crimping or otherwise attaching single-piece Nitinol (or any other material) extension, a distal end of extension 1603 with wire diameter equal to the body of the extension and a small tapered portion to eliminate any step-off between the needle and extension, the body of extension 1604, a suture-attachment portion 1605 of the extension, and a suture thread 1606. The embodiment shown in FIG. 16 is an iteration almost identical in description to the embodiment shown in FIG. 15. The only difference lies in the attachment of the extension to the needle, which has no step-off. In this iteration, the extension includes a portion that increases in diameter to match the diameter of the needle (at the end of its tapered section) and then small tapers back to the wire diameter of the extension body. This serves to eliminate the small step-off between the needle and the extension that is present in the embodiment shown in FIG. 15. The connection of the needle to the extension and the connection of the extension to the suture can take the forms of all iterations disclosed herein.

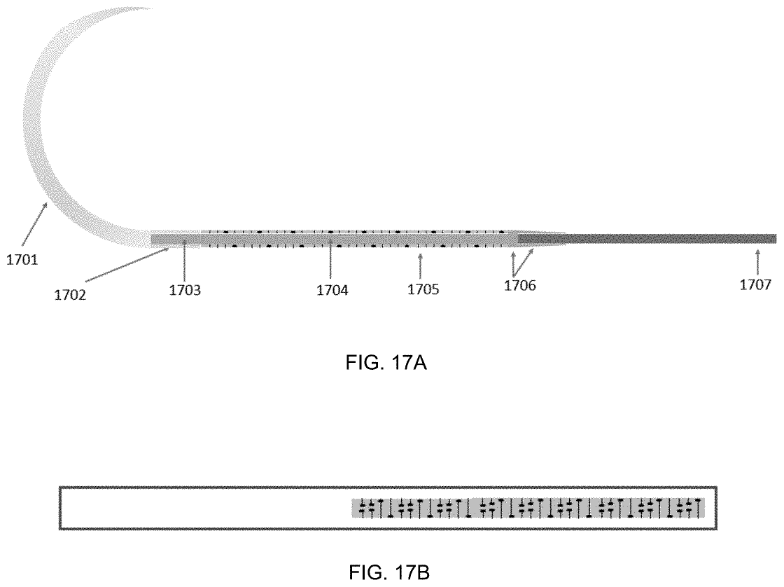

With reference to FIG. 17A, another embodiment of a curved suturing needle 1701 is shown. Curved suturing needle 1701 comprises a tapered extension attachment portion 1702 for crimping or otherwise attaching single-piece Nitinol (or any other material) extension, a distal end of the nitinol component 1703, the body of the nitinol component 1704, a sleeve 1705 surrounding nitinol component, a proximal end of nitinol component 1706 comprising a sleeve retaining portion and a suture-attachment portion, and a suture thread 1707. This iteration comprises an extension having multiple components. FIG. 17B shows a “dog bone” pattern laser-cut tube sheath (or sleeve) surrounding the nitinol wire. The sleeve (made of any suitable metal, metal alloy (nitinol or other), polymer or any other suitable material or any combination of materials thereof) having any given pattern of cuts, indentations or any other patterning (produced by laser-cutting, molding, additive manufacturing, 3D-printing or any other method) is included herein. The sleeve may or may not be welded or otherwise bonded or attached to the proximal end of the needle. In this iteration, the distal end and the body of the nitinol component has a wire diameter smaller than the wire diameter of the needle, as previously described (for attachment to the needle and for flexibility purposes), but the proximal end of the nitinol component increases in wire diameter to match the diameter of the surrounding sleeve and needle wire diameter. This creates a retaining structure to maintain the sleeve captive between the proximal end of the nitinol component and the needle without the need for an additional, separate component to serve this function. The proximal end of the nitinol component (the trailing end connecting to the suture) comprises a tapered suture-attachment portion for which multiple iterations have been described (FIG. 13B-F and all their combinations thereof) and all these iterations apply to all the multi-component extensions disclosed herein.

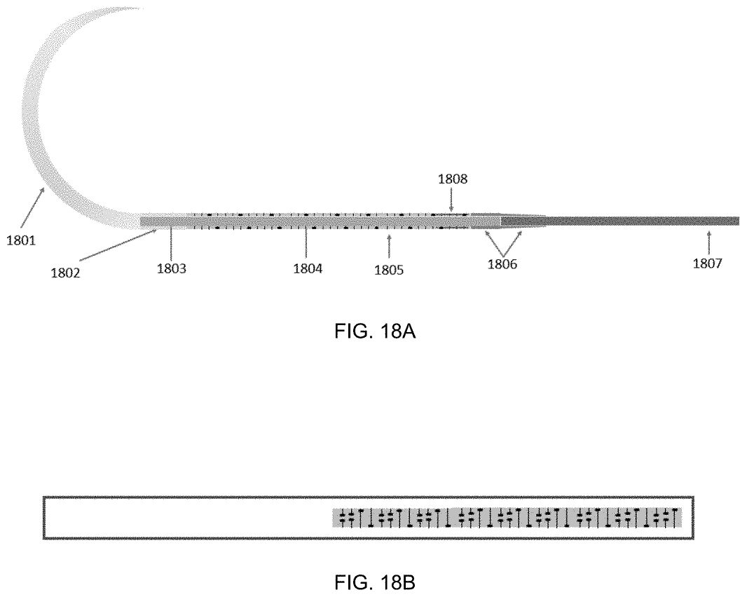

With reference to FIG. 18A, another embodiment of a curved suturing needle 1801 is shown. Curved suturing needle 1801 comprises an extension attachment portion 1802 for crimping or otherwise attaching single-piece Nitinol (or any other material) extension, a distal end of the nitinol wire 1803 with wire diameter, the body of the nitinol component 1804, a sleeve 1805 surrounding nitinol component, a separate component 1806 to crimp proximal end of nitinol wire and comprising a tapered suture-attachment portion, and a suture thread 1807, and a crimp 1808 securing sleeve to nitinol wire to avoid free rotation of the sleeve around wire. This iteration comprises an extension having multiple components. FIG. 18B shows a “dog bone” pattern laser-cut tube sheath (or sleeve) surrounding the nitinol wire. The sleeve (made of whatever metal, metal alloy (nitinol or other), polymer or any other suitable material or any combination of materials thereof) having any given pattern of cuts, indentations or any other patterning (produced by laser-cutting, molding, additive manufacturing, 3D-printing or any other method) is included as part of this disclosure. The sleeve may or may not be welded to the proximal end of the needle. The proximal end of the nitinol wire is crimped (or otherwise attached) to a separate component that also comprises a tapered suture-connection portion (for crimping or otherwise attaching the suture). The tapered suture-attachment portion for which multiple iterations have been described (FIG. 13B-F and all combinations thereof) and all these iterations apply to all the multi-component extensions disclosed herein.

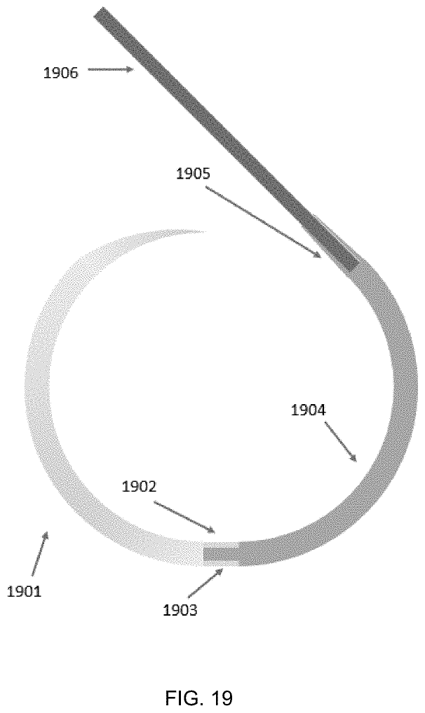

With reference to FIG. 19, another embodiment of a curved suturing needle 1901 is shown. Curved suturing needle 1901 comprises an extension attachment portion 1902 for crimping or otherwise attaching single-piece Nitinol (or any other material) extension, a portion of the extension 1903 with a smaller diameter to facilitate crimping (or otherwise attaching) the extension to the needle, the body of the curved single-piece nitinol extension 1904 having the same diameter as wire diameter of the needle, a suture-connecting/attachment portion 1905 of the single-piece nitinol extension having a tapered configuration, and a suture thread 1906. This iteration shows an extension (single-piece or multi-component) that is curved (as an example) to facilitate the needle returning back to a needle driver (roller-based needle driver, traditional needle driver needle driver or any other device used to drive a needle through live tissues or inanimate objects). An exemplary application is to allow for continuous driving of a needle by having the needle cycle through a roller-based needle driver, wherein the rollers drive past the end of the needle and re-engage its tip to continue driving the needle. Although the exemplary embodiment shown here depicts a curved configuration for the extension, any and all other non-straight (non-linear) extension configurations such as J-shaped, S-shaped, L-shaped, compound curved, angled, multi-angled, spiral, corkscrew/helical or having any other non-linear shaped, or an extension having any combination or permutation of any and all of these shapes can be used in the embodiments disclosed herein. In certain embodiments, all these extension variations can be magnetic. The description disclosing non-linear extension configurations is meant to apply to all single-piece extension embodiments disclosed herein, as well as all multi-component extension embodiments disclosed herein, and the different combinations and permutations of these concepts thereof, including needles of any size, wire diameter, length, curvature, material, or shape. In the embodiments disclosed herein of an extension with any kind of curved component (simple curve or compound curve) the length and extension of the arc can be such as to be any amount of degrees from 0 degrees to more than 360 degrees, to complete a circular, elliptical or any other type of cycloid path that make the suture or suture-attachment portion of the extension to meet (or otherwise come in contact with or close proximity to) any part of the needle, needle tip or another point of the extension. The non-linear extension may curve or otherwise extend non-linearly in the same plane as the needle or the extension, it may extend or curve in any plane or plurality of planes different to the needle or extension, or it may curve/extend in and out of any plane or plurality of planes relative to the needle or the extension in any combination or permutation of planar orientations. The non-linear extension can be of any length. All the embodiments of needles with smooth and non-smooth surfaces as well as all the variations for attachment of an extension to a needle (including a straight needle) and all the permutations describing different combinations of needle configuration and suture material (barbed, non-barbed, monofilamentous, braided, absorbable, non-absorbable, and or as otherwise described herein) can be used to describe all the embodiments comprising a single-piece nitinol extension, multi-component extensions in their linear and non-linear iterations. The cross-section of any single-piece or multi-component extension and the cross-section of the needle can each be (independent of each other) round, oval, square, pentagonal, hexagonal or any other shape. All of these provisions also apply to all the other embodiments disclosed herein.

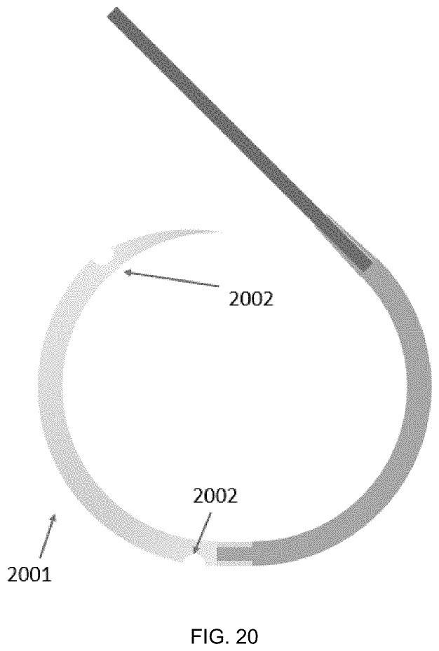

With reference to FIG. 20, another embodiment of a curved suturing needle 1901 is shown. Curved suturing needle 2001 is shown as comprising indentations or areas with a geometry meant for engaging with any structure meant to drive or otherwise direct the needle in any direction 2002, and the remaining components in the curved suturing needle shown in FIG. 19. This iteration describes the same needle iteration of FIG. 19 but includes the presence of at least an area (or plurality of areas) having any kind of geometry or feature meant to allow the needle to engage with any structure that would drive, exert a force upon or otherwise direct the needle in any direction. In this exemplary embodiment, these areas take the shape of indentations that are of a generally round shape. In certain embodiments, these areas for needle engagement may include indentations with a square, triangular, oval, polygonal (any number of sides), curved, compound curved, other regular shape or irregular shape. Moreover, these areas for needle engagement may not be indentations but may be protrusions, flanges, hooks or even areas having different textures or material compositions to promote engagement of the needle with any structures that would drive, exert a force upon or otherwise direct the needle in any direction. In certain embodiments, this area (or plurality of areas) for needle engagement may be magnetic. The structures that may engage the needle may include pawls, hooks, surfaces that may interfere and therefore exert a force upon any protrusion or other feature of the needle, textured or otherwise specifically prepared or modified surfaces conducive to driving, exerting a force upon or otherwise directing the needle or any other structures that may engage the needle through either direct mechanical means or through magnetism. In certain embodiments, the needle extension, as well as the needle, a portion or plurality of portions of the needle or any combination thereof may be magnetic or magnetizable.

Features or functionality described with respect to certain example embodiments may be combined and sub-combined in and/or with various other example embodiments. Also, different features and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, can be at least partially performed via at least one entity or actor in any manner.

As used herein, a term “about” or “substantially” refers to a +/−10% variation from a nominal value/term. Such variation is always included in any given value/term provided herein, whether or not such variation is specifically referred thereto.

In particular, the presently disclosed suture needles can be used for an open surgery, a minimally invasive surgery, a laparoscopic surgery, or an end effector robotic surgery. As such, the presently disclosed suture needles can be used for manual surgery or automated surgery. Some examples of surgeries where the presently disclosed suture needles can be employed include laparoscopic surgery, robotic surgery, video-assisted or unassisted thoracoscopic surgery, arthroscopic surgery, natural orifice surgery, endoscopic surgery, gynecologic surgery, cardiac surgery, colorectal surgery, pulmonary surgery, gastric bypass surgery, hysterectomy surgery, dental surgery, urological surgery, brain surgery, fetal surgery or bariatric surgery, or among many others in human (e.g., between newborn until 120 years old, male, female) or animal (e.g., mammal, birds, fish, land animals) applications. However, note that the presently disclosed suture needles can also be applied to non-medical applications, such as garment making, fabric stitching, knot applications, sowing, shoe making, or others.

Any component described herein can include a material suitable for a medical use. The material can be, flexible, elastic, or resilient. The material can be suitable to be disinfected, sterilized, or sanitized, which can be with a hot steam, an autoclave, or others. For example, the material can include plastic, metal, rubber, shape memory, fabric, foam, or others.

The device and system of the present disclosure has been described with specific reference to certain drawings and various embodiments, but may, however, be embodied in many different forms and should not be construed as necessarily being limited to only embodiments disclosed herein. Rather, these embodiments are provided so that this disclosure is thorough and complete, and fully conveys various concepts of this disclosure to skilled artisans.

Note that various terminology used herein can imply direct or indirect, full or partial, temporary or permanent, action or inaction. For example, when an element is referred to as being “on,” “connected” or “coupled” to another element, then the element can be directly on, connected or coupled to the other element or intervening elements can be present, including indirect or direct variants. In contrast, when an element is referred to as being “directly connected” or “directly coupled” to another element, there are no intervening elements present.

Likewise, as used herein, a term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances.

Similarly, as used herein, various singular forms “a,” “an” and “the” are intended to include various plural forms as well, unless context clearly indicates otherwise. For example, a term “a” or “an” shall mean “one or more,” even though a phrase “one or more” is also used herein.

Moreover, terms “comprises,” “includes” or “comprising,” “including” when used in this specification, specify a presence of stated features, integers, steps, operations, elements, or components, but do not preclude a presence and/or addition of one or more other features, integers, steps, operations, elements, components, or groups thereof. Furthermore, when this disclosure states that something is “based on” something else, then such statement refers to a basis which may be based on one or more other things as well. In other words, unless expressly indicated otherwise, as used herein “based on” inclusively means “based at least in part on” or “based at least partially on.”

Additionally, although terms first, second, and others can be used herein to describe various elements, components, regions, layers, or sections, these elements, components, regions, layers, or sections should not necessarily be limited by such terms. Rather, these terms are used to distinguish one element, component, region, layer, or section from another element, component, region, layer, or section. As such, a first element, component, region, layer, or section discussed below could be termed a second element, component, region, layer, or section without departing from this disclosure.

Also, unless otherwise defined, all terms (including technical and scientific terms) used herein have the same meaning as commonly understood by one of ordinary skill in an art to which this disclosure belongs. As such, terms, such as those defined in commonly used dictionaries, should be interpreted as having a meaning that is consistent with their meaning in a context of a relevant art and should not be interpreted in an idealized or overly formal sense unless expressly so defined herein.

In addition, features described with respect to certain example embodiments may be combined in or with various other example embodiments in any permutational or combinatory manner. Different features or elements of example embodiments, as disclosed herein, may be combined in a similar manner. The term “combination”, “combinatory,” or “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context or the prior art.

Although preferred embodiments have been depicted and described in detail herein, skilled artisans know that various modifications, additions, substitutions and the like can be made without departing from the spirit of this disclosure. As such, these are considered to be within the scope of the disclosure, as defined in the following claims.

1. A suturing needle comprising:
a needle tip portion having a needle tip at a distal end for piercing tissue;
a needle body having a distal end connected to a proximal end of the needle tip portion;
a hollow thread attachment component having a distal end attached to a proximal end of the needle body, the thread attachment component comprising a viewing port,
a hollow extension ramp portion having a distal end and a tapered portion at proximal end, the distal end attached to a proximal end of the thread attachment component; and
a suture thread attached to the thread attachment component.

2. The suturing needle of claim 1, wherein the distal end of the ramp has a diameter equal to the needle body diameter, and the proximal end of the ramp has a diameter equal to a suture thread diameter, and wherein the tapered portion provides a smooth transition from the proximal end of the ramp portion to the distal end thereof.

3. The suturing needle of claim 1, wherein the suture thread is attached to the thread attachment component by swaging when the suture thread is visible through the viewing port.

4. The suturing needle of claim 1, wherein the suture thread is mono-filamentary or multi-filamentary.

5. The suturing needle of claim 1, wherein the suture thread is bioabsorbable.

6. The suturing needle of claim 1, wherein the suture thread comprises at least one region of tissue retaining structures.

7. The suturing needle of claim 6, wherein the tissue retaining structures are barbs.

8. The suturing needle of claim 6, wherein the at least one region of tissue retaining structures are distal from the tapered portion of the ramp portion.

9. The suturing needle of claim 1, wherein the needle tip portion is formed to become gradually thicker from the needle tip to the proximal end thereof.

10. The suturing needle of claim 1, wherein the needle body is straight.

11. The suturing needle of claim 1, wherein the needle body curved.

12. The suturing needle of claim 1, wherein the needle body is formed to have a substantially circular cross-section.

13. The suturing needle of claim 1, wherein the needle body is formed to have a substantially circular cross-section having a smooth profile.

14. The suturing needle of claim 1, wherein the needle body is formed to have a substantially circular cross-section having a non-smooth profile.

15. The suturing needle of claim 14, wherein the non-smooth profile comprises a plurality of equally spaced circumferential indentations extending longitudinally on the curved needle body.

16. The suturing needle of claim 15, wherein the non-smooth profile comprises from 4 to 16 equally spaced circumferential indentations forming ridges having a convex shape.

17. The suturing needle of claim 15, wherein the circumferential indentations extend along a full length of the curved needle body.

18. The suturing needle of claim 17, wherein the circumferential indentations are angled on the curved needle body to form a spiral thereon.

19. The suturing needle of claim 18, wherein the spiral is right-handed or left-handed.

20. The suturing needle of claim 18, comprising at least two regions of circumferential indentations having different angles on the curved needle body.

21. The suturing needle of claim 1, wherein the needle body is formed to have a substantially hexagonal or otherwise non-circular polygonal cross-section.

22. The suturing needle of claim 1, further comprising a hollow flexible extension component having a distal end and a proximal end, the distal end connected to the proximal end of the thread attachment component, and the proximal end attached to the distal end of the tapered extension ramp portion.

23. The suturing needle of claim 22, wherein the hollow flexible extension component has a diameter equal to the needle body diameter.

24. The suturing needle of claim 22, wherein the hollow flexible extension component comprises laser cuts.

25. The suturing needle of claim 24, wherein the laser cuts form a spiral.

26. The suturing needle of claim 22, wherein the hollow flexible extension component comprises a cable.

27. The suturing needle of claim 26, wherein the cable forms a spiral.

28. The suturing needle of claim 22, wherein the hollow flexible extension component comprises a wire.

29. The suturing needle of claim 28, wherein the wire forms a spiral.

30. A method of suturing tissues using a roller needle driver, the method comprising the steps of:
(a) providing a suture needle comprising: a needle tip portion having a needle tip at a distal end for piercing tissue; a needle body having a distal end connected to a proximal end of the needle tip portion; a hollow thread attachment component having a distal end attached to a proximal end of the needle body, the thread attachment component comprising a viewing port, a ramp portion having a distal end and a tapered portion at proximal end, the distal end attached to a proximal end of the thread attachment component; and a suture thread attached to the thread attachment component;
(b) grasping, between rollers of the roller needle driver, the suture thread or the tapered portion of the ramp portion distal from the needle body;
(c) loading the needle so that the rollers are positioned on the needle body without opening the rollers;
(d) driving the needle through a tissue being subjected to suture;
(e) moving the needle through the tissue without releasing the needle from the tissue;
(f) grasping the suture needle at an end exiting from the tissue through which the needle was moved; and
(g) repeating steps (d)-(f) until completion of the suturing process.

31. A suturing needle comprising:
a needle tip portion having a needle tip at a distal end for piercing tissue;
a needle body having a distal end connected to a proximal end of the needle tip portion;
an extension component having a distal end attached to a proximal end of the needle body, the distal end of the extension component comprising a cross section smaller than a cross section of the proximal end of the needle body, the extension component having a hollow proximal end; and
a suture thread positioned in and coupled to the hollow proximal end of the extension component.

32. A device comprising:
An elongated, flexible, metal or metallic component comprising:
a distal end configured for coupling to a hollow proximal end of a suturing needle, wherein the distal end comprises a cross section smaller than a cross section of the hollow proximal end of the suturing needle;
a middle portion; and
a proximal end, wherein the middle portion extends between the distal end and the proximal end, wherein the proximal end comprises a hollow portion, and wherein the hollow portion is configured for coupling to a suture thread.
